(19) United States
(12) Patent Application Publication
(10) Pub. No.: US 2008/0167281 A1
(43) Pub. Date: Jul. 10, 2008

(54) Combination Otic Formulation

(76) Inventor: David M. PRESTON, Chapel Hill, NC (US)

Correspondence Address: HUTCHISON LAW GROUP PLLC, PO BOX 31686, RALEIGH, NC, 27612, US

(21) Appl. No.: 11/620,374

(22) Filed: Jan. 5, 2007

(51) Int. Cl. A61K 31/57; A61K 31/24; A61P 27/16

(52) U.S. Cl. 514/171; 514/537

(57) ABSTRACT

A therapeutic composition and methods for the treatment and prevention of otic disorders in humans and animals comprising an analgesic agent, an anesthetic agent, a combination of an astringent and an anti-infective are disclosed. The composition and method provides for contacting the tympanic membrane without substantially penetrating the tympanic membrane. The composition and method may further comprise polycosanols, a steroidal anti-inflammatory, aloe and mixtures thereof.

### FIELD OF THE INVENTION

The subject of this invention relates to compositions and methods of treatment and prevention of pathological symptoms of the external and middle ear, the composition preferably being non-invasive to the tympanic membrane in such a manner so as to not contribute to the build up of fluid behind the tympanic membrane.

### BACKGROUND

Otic disorders are believed to rank second only to the common cold as the most frequent illness among children in the United States. Most otic disorders are responses to inflammation from infections, allergic reactions, or trauma to the ear. An otic infection may be of bacterial, fungal or viral origin. Determination of the precise etiology may not practical since the causative organism may be difficult to isolate and culture. A common otic disorder includes otitis. Otitis is a non-specific term that describes a symptom and indicates an inflammation of the ear.

The ear is anatomically divided into the external, middle and inner ear. The external ear consists of the auricle and the external canal, a tube like structure that ends at the eardrum (tympanic membrane).

The middle car or tympanic cavity, is an air-filled cavity in the temporal bone that contains three small bones (malleus, incus and stapes) that transfer sound from the tympanic membrane to the oval window of the inner ear. The eustachian tube connects the middle ear with the back of the nasopharynx. The tube's function is to allow air to enter the middle ear and maintain equal atmospheric pressure on both sides of the tympanic membrane. An inflammation of the lining of the eustachian tube, due to infection or allergies, causes the tube to close and either creates a vacuum or the accumulation of fluid in the middle ear resulting in otitis media.

The inner ear is concerned with the reception of sound and balance. Since the organs for hearing and balance are within the same compartment of the temporal bone, they are often affected by the same disorders. Inflammation in the middle ear frequently causes inflammation in the inner ear resulting in disorders of hearing and balance. It is important to treat the inflammation as soon as possible to reduce the sequella of hearing loss, tinnitus, facial nerve palsy, mastoiditis, labyrinthitis, vertigo, and possible encephalitis.

Normally, cerumen (ear wax) and the acid pH of the external auditory canal protect the ear from infection. The canal can become inflamed and infected by trauma to the epithelium lining of the canal, for example, after attempts to remove cerumen or entrapped water. Because the epithelium of the external auditory canal is tightly attached to the underlying bone or cartilage, even a little swelling produces a great deal of pain. Trauma to the canal may cause the epithelium to become macerated and susceptible to infection. The macerated epithelial cells form a red and scaly dermatitis that may encroach on the epithelium of the tympanic membrane. In one mode of treatment, a cellulose tampon (Pope ear wick) may be inserted into the auditory canal and moistened with antibiotics and drying-medications or steroids to control the infection and to relieve the swelling. Fungal infections of the external auditory canal, which may result, may be resolved by restoring the acidic pH of the external ear.

Otitis media is a painful inflammation of the middle ear. Acute otitis media is usually accompanied by fever, swelling, inflammation of the tympanic membrane and considerable pain. Otitis media develops when bacteria or viruses, usually associated with colds or sore throats, make their way up the eustachian tube, from the upper part of the throat behind the nose to the middle ear. When fluid accumulates in the middle ear the condition is known as otitis media with effusion or “glue ear.” This condition may lead to hearing loss and may affect developmental learning and language skills.

Nearly 70 percent of U.S. children will develop otitis media by age 2. Otitis media is a frequent problem in children because the eustachian tube is shorter, wider, and more horizontal than in adults. This anatomical difference may facilitate the spread of pathogens from the nasopharnyx to the middle ear, which may result in infection and a painful inflammatory response in the mucosal tissue of the middle ear. Many children will outgrow their susceptibility to the infection by age 5. Over half of those who experience acute otitis media will have repeated episodes and the condition may become chronic. Otitis media may be the most common cause of hearing loss in the U.S. and may represent a significant disability interfering with childhood learning processes. It is believed that otitis media accounts for over 35 percent of all visits to pediatricians each year and represents more than $3.5 billion in U.S. health care costs annually.

Otitis externa (swimmer's ear) is an inflammation of the external canal which occurs in hot, humid weather, after cooling off in the pool and by those who enjoy aerobic exercises. Untreated or chronic otitis externa may also cause an inflammation of the tympanic membrane and initiate an cascade-like inflammation of the middle and inner ear.

Subjects suffering from an otic disorder would, historically, have had to take multiple separate medications to treat both the underlying disorder and the associated pain and inflammation.

In view of the aforementioned, it is desirous to provide a topical composition which specifically directs therapeutically effective combination of agents simultaneously to the external and middle to treat and prevent the effects of inflammation, pain and infection while minimizing or preventing degradation of tympanic membrane quality, as herein described.

### SUMMARY

Methods and compositions for the treatment and prevention of pathological symptoms of the outer or inner ear with a composition comprising an analgesic agent, an anesthetic agent, and an astringent and an anti-infective system are described. In embodiments, a therapeutic composition for the treatment and prevention otic disorders in humans and animals is provided. The composition consists essentially of benzocaine, antipyrine, aluminum acetate, acetic acid and optionally polycosanol, an anti-inflammatory agent or aloe or combinations thereof.

In another embodiments, a therapeutic composition for the treatment and prevention of otic disorders in humans and animals is provided. The composition comprises an analgesic agent, an anesthetic agent, and a combination of an astringent and an anti-infective, where the composition is substantially free of COX-2 inhibitors.

In other embodiments, a method for the treatment and prevention of otic disorders in humans and animals is provided. The method comprises administering a composition comprising an analgesic agent, an anesthetic agent, a combination of an stringent and an anti-infective, optionally with polycosanol, a steroidal anti-inflammatory or aloe, the composition being substantially free of COX-2 inhibitors where the composition provides for contacting of the tympanic membrane without substantially penetrating the tympanic membrane.

In embodiments, a method for the treatment and prevention of otic disorders in humans and animals in need thereof is provided. The method comprises contacting, without substantially penetrating, the tympanic membrane of a subject with a composition consisting essentially of an analgesic agent, an anesthetic agent, a combination of an astringent and an anti-infective, polycosanol, and a carrier. The carrier allows administration by drops placed into the external ear canal, optionally in combination with an anti-inflammatory agent or aloe or combinations thereof.

### DETAILED DESCRIPTION

In accordance with the embodiments herein disclosed, otic disorders and otic disorder-related complications may be treated and prevented in a subject by administering to the subject an analgesic agent, an anesthetic agent, and a combination of an astringent and an anti-infective agent.

An example of an otic disorder possibly triggered by an infectious agent is otitis media. A bacterial otic infection may arise as a primary infection or it may also arise secondary to a viral infection. However, inflammation in the middle ear mucosa, typically caused by bacterial pathogens, may be the primary event in the middle ear predisposing the development of the otic disorder, otitis media with effusion. Accordingly, the methods and compositions of the present invention comprise a treatment and prevention of the underlying otic disorder, and the corresponding pain and inflammation in a subject who may already have or who may be predisposed to developing an otic disorder.

The methods and compositions of the present invention comprise the prevention or treatment of otic disorders and otic disorder-related disorders in humans and animals. More preferably, the methods and compositions of the present invention comprises the prevention and treatment of the otic disorders including otitis media, typanitis, myringitis, otitis media with effusion, otitis externa and labyrinthitis. Even more preferred, the methods and compositions of the present invention comprises the prevention and treatment of the otic disorders otitis media and otitis externa. The methods and compositions of the present invention may also be useful to reduce, eliminate or retard, in subjects, the development of complications associated with otic disorders, such as, for example, hearing loss, brain abscess, meningitis and facial paralysis, which may eventually arise from having a chronic or recurring otic disorder.

Thus, the compositions described herein may be useful, for example, to reduce or eliminate such otic disorder symptoms as, for example, otic pain, and inflammation in a subject suffering from such symptoms. The compositions would also be useful to prevent the occurrence of such symptoms.

In embodiments, the compositions and methods of treating an otic disorder comprise administration of an analgesic agent, an anesthetic agent, and a combination of an astringent and an anti-infective agent. Such compositions may provide for the prevention and treatment of otic disorders and otic disorder-related complications and may provide an unexpectedly effective treatment and preventative therapy. Such administration may be effective for improving the symptoms of otic disorders and otic disorder-related complications while avoiding or reducing certain disadvantages of current treatments.

Furthermore, the administration of an analgesic agent, an anesthetic agent, and a combination of an astringent and an anti-infective agent may be superior to the use of either agent or sub-combination of agents alone. The use of an analgesic agent, an anesthetic agent, and a combination of an astringent and an anti-infective agent may also be useful for decreasing the required number of separate dosages, thus, potentially improving patient compliance. For example, the combination therapy may be effective for lowering the dosages of otic agents that are normally prescribed as a treatment. The administration of lower dosages of conventional active agents may provide a reduction in side effects corresponding to such conventional agents or sub-combinations of such agents. Moreover, the compositions and methods herein may provide for minimizing or preventing degradation of tympanic membrane quality of the subject. As used herein, the term “minimizing or preventing degradation of tympanic membrane quality” refers to maintaining the tympanic membrane of the subject in a condition substantially similar to a condition just prior to preventing or treating the subject by the method herein described.

Compositions comprising an analgesic agent, an anesthetic agent, and a combination of an astringent and an anti-infective agent may be useful for improving otic disorder symptoms, shortening recovery times and for reducing the dosages of otic agents that are normally required. Reduced dosages of otic agents are beneficial where normal dosages often exhibit harmful side effects, for example, with such otic agents as acetic acid, corticosteroids and antibiotics. Side effects from acetic acid may include compromise of the tympanic membrane, rash, stinging and burning sensations and general discomfort. Side effects from corticosteroid use can include osteoporosis, susceptibility to bruising, diabetes, cataracts, glaucoma, high blood pressure, infections and weight gain.

A composition and method of treatment for an otic disorder comprising an analgesic agent, an anesthetic agent, and a combination of an astringent and an anti-infective agent may be efficacious for impairing the process of inflammation within the ear, thus preventing or treating otic disorders and thereby otic disorder-related complications. Moreover, an analgesic agent, an anesthetic agent, and a combination of an astringent and an anti-infective agent may provide synergistic effects, which reduce the symptoms associated with otic disorders and otic disorder-related complications to a greater extent than would be expected on the basis of the use of either agent or sub-combination of agents alone. The term “synergistic” refers to the combination of an analgesic agent, an anesthetic agent, and a combination of an astringent and an anti-infective agent as a combined therapy having an efficacy for the prevention and treatment of otic disorders that is greater than the sum of their individual or sub-combination effects.

The synergistic effects of embodiments herein described may comprise additional unexpected advantages for the treatment and prevention of otic disorders. Such additional advantages include, but are not limited to, lowering the required dose of otic agents, reducing the side-effects of otic agents, and rendering those agents more tolerable to subjects undergoing otic disorder therapy.

The composition described herein comprises an analgesic agent, an anesthetic agent, and a combination of an astringent and an anti-infective agent that may be useful for the purpose of preventing and treating otic disorders and otic disorder-related complications in a subject that is in need of such prevention or treatment.

As used herein, “pharmacologically active agent” or “active agent” or “agent” are used interchangeably and refer to a compound or composition of matter which, when administered to a human or animal induces a desired pharmacologic and/or physiologic effect by local and/or systemic action.

The anesthetic may include any local anesthetics known in the art (see, for example, those listed in the Merck Index, 13th ed., 2001 herein incorporated by reference). Local anesthetics may include, for example, compounds such as benzocaine, bupivicaine, butamben picrate, carbocaine, chloroprocaine, cocaine, dibucaine, etidocaine, etidocaine, fenamates, lidocaine, mepivacaine, oxicams, pramoxine, prilocaine, pyrazolone derivatives, pyrrolealkanoic acids and tetracaine, and mixtures thereof. Preferably, the anesthetic is a topical anesthetic. More preferably, the topical anesthetic is benzocaine. The anesthetic may be present in the composition in a range of concentrations from about 0.01% to about 10% weight/volume (w/v) of the composition.

Two or more local anesthetics with different pharmacokinetics with respect to the etiological and pathological symptoms of otic disorders may be combined in the composition, with the individual local anesthetics being present in concentrations such that the overall concentration of the local anesthetics in the composition does not exceed 10% w/v of the composition.

The term “analgesic” refers to compounds that relieve pain and achieve analgesia. Analgesics include, for example, antipyretics, arylpropionic acid derivatives and salicylic acid derivatives. In embodiments, the analgesic is antipyrine and its salts, hereinafter referred to as antipyrine.

The analgesic may be present in the composition in a range of concentrations from about 0.01% to about 10% w/v of the composition. Two or more analgesics with different pharmacokinetics with respect to the etiological and pathological symptoms of otic disorders may be combined in the composition, with the individual analgesic being present in concentrations such that the overall concentration of the analgesics in the composition does not exceed 10% w/v of the composition.

The term “astringent” refers to a compound that tends to shrink or constrict tissue, usually locally after topical application. For example, astringent includes aluminum acetate, zinc acetate, formic acid, boric acid and the like. In embodiments, the astringent is aluminum acetate. Certain compounds may function as both an astringent and as an anti-infective, for example, acetic acid. When such compounds, which may function as both an astringent and as an anti-infective, are used in the composition as described herein as the astringent, it is generally preferred that another compound be used as the anti-infective.

The astringent may be present in the composition in a range of about 0.01% to about 10% w/v of the composition. Two or more astringents with different pharmacokinetics with respect to the etiological and pathological symptoms of otic disorders may be combined in the composition, with the individual astringent being present in concentrations such that the overall concentration of the astringents in the composition does not exceed 10% w/v of the composition.

The term “anti-infective” is used interchangeably with the terms “antibacterial” or “antiseptic” and refers to any chemical of natural or synthetic origin that has the effect to kill or inhibit or suppress the growth of biological cells. Anti-infectives may kill or suppress the growth of bacteria, which are susceptible to low pH environments and include compounds such as, for example, acetic acid, boric acid, gentian violet, hydrogen peroxide, carbamide peroxide, chlorhexidine, mercurochrome, povidone iodine, polyhyroxine iodine, and cresylate, and mixtures thereof. Certain compounds may function as both an anti-infective and as an astringent, for example, aluminum acetate. When such compounds, which may function as both an anti-infective and as an astringent, are used in the composition as described herein as the anti-infective, it is generally preferred that another compound be used as the astringent.

In embodiments, the anti-infective compounds include low molecular weight organic acids and their metallic salts. In embodiments, the anti-infective is acetic acid, benzalkonium chloride and benzethonium chloride. The anti-infective may be present in the composition in a range of concentrations from about 0.01% to about 4% w/v of the composition. In embodiments, the anti-infective is acetic acid is present in a concentration of less than 2% w/v of the composition, preferably 1.75% w/v of the composition.

Two or more anti-infectives with different pharmacokinetics with respect to the etiological and pathological symptoms of otic disorders may be combined in the composition, with the individual anti-infective being present in concentrations such that the overall concentration of the anti-infectives in the composition does not exceed 4% w/v of the composition. For example, acetic acid and benzalkonium chloride and/or benzethonium chloride may be combined in the composition, provided that the acetic acid is present in a concentration such that the overall amount of acetic acid is less than 2% w/v of the total composition.

The herein described composition for preventing or treating otic disorders and otic disorder-related complications may comprise an analgesic agent, an anesthetic agent and a combination of an astringent and an anti-infective agent where the composition is substantially free of COX-2 inhibitors. The term “COX-2 inhibitors” refers generally to compounds that directly target the COX-2 inducible enzyme but substantially do not target the COX-1 constitutive enzyme. The term COX-2 inducible enzyme, refers to the cyclooxygenase enzyme generally undetectable in most normal tissues but becoming abundant in activated macrophages and other cells at sites of inflammation.

Optional pharmacologically active components may be combined with the composition herein described and used in combination with an analgesic agent, an anesthetic agent, and a combination of an astringent and an anti-infective agent. Thus, the composition herein disclosed for preventing or treating otic disorders and otic disorder-related complications may comprises an analgesic agent, an anesthetic agent, a combination of an astringent and at least one optional pharmacologically active otic agent.

As used herein, “pharmacologically active otic agent” or “active otic agent” or “otic agent” are used interchangeably and refers to any compound having a therapeutic effect on an otic disorder or an otic disorder agent, whether in vivo or in vitro, over any duration of time other than a compound or composition of matter that is an analgesic agent, an anesthetic agent, an astringent or an anti-infective agent. This therapeutic effect can occur via bacterial and/or viral growth suppression, inflammation reduction, eustachian tube dilation, or by any other mechanism. The otic agent includes, but is not limited to, at least one compound selected from the group consisting of polycosanol, anti-inflammatory agents, aloe, antibiotics, anticholinergics, antihistamines, decongestants and mixtures thereof.

Examples of preferred classes of otic agents capable of being used in combination with an analgesic agent, an anesthetic agent, and a combination of an astringent and an anti-infective agent include, but are not limited to, one or more of polycosanols, steroidal anti-inflammatory agents, aloe and combinations thereof. Otic agents that may assist in reducing the pain a subject suffers from an otic disorder includes the polycosanols. The term “polycosanols” refers generally to an extract of plant/insect waxes, the waxes comprising from about 24 to about 36 carbon atoms. Polycosanol and polycosanols, as used herein, include one or more fatty alcohols extracted and/or derived from the waxes of plants such as sugar cane, rice bran and yams, as well as beeswax and Ericerus Pela wax secreations. Polycosanol as used herein may comprise one or more, preferably two or more, more preferably three or more of the fatty alcohols octanosol, triacontanol (melissyl alcohol or myricyl alcohol), behenyl alcohol, lignoceryl alcohol, ceryl alcohol, 1-heptacosanol, 1-nonacosanol, 1-dotriacontanol, and geddyl alcohol. Polycosanols includes extracts that may be subsequently treated such as by saponification or hydrolysis and then fractionated to produce a complex mixture that may be artificially enriched with a mixture of higher primary aliphatic alcohols. The polycosanol may be present in the composition in a range of concentrations from about 0.01% to about 50% w/v of the composition.

Otic agents that may assist in reducing the pain or inflammation a subject suffers from an otic disorder include anti-inflammatory agents. Anti-inflammatory agents may include steroidal anti-inflammatory agents. Steroidal anti-inflammatory agents may include the corticosteroids. Corticosteroid otic agents include, for example, alclometasone, amcinonide, betamethasone, betamethasone, betamethasone valerate, clobetasol, clocortolone, cortisol, cortisone, desonide, desoximetasone, dexamethasone, diflorasone, flumethasone, fluocinolone acetonide, fluocinonide, fluorometholone, fluprednisolone, flurandrenolide, flurandrenolone acetonide, fluticasone, halcinonide, halobetasol, hydrocortisone, methylprednisolone, mometasone, prednicarbate, prednisolone, prednisone and triamcinolone, and mixtures thereof. In embodiments, the corticosteroid otic agent is hydrocortisone. The steroidal anti-inflammatory agents may be present in the composition in a range of concentrations from about 0.01% to about 5% w/v of the composition.

Otic agents that may assist in reducing the pain a subject suffers from an otic disorder include aloe. Aloe includes a portion of expressed juice of the leaves of non-toxic plants species from the genus Asphodelaceae. Species of aloe includes A. vera, A. vulgaris, A. socotrina, A. chinensis, and A. perryi, and further includes commercially available aloes such as Barbadoes, Socotrine, Hepatic, Indian, and Cape aloes. The term “aloe” is used herein interchangeably with “aloe vera.” The aloe may be present in the composition in a range of concentrations from about 0.01% to about 10% w/v of the composition.

In embodiments, a therapeutic otic composition comprises 0.01% 10% (w/v) benzocaine, 0.01%-10% (w/v) antipyrine, 0.01%-10% (w/v) aluminum acetate, less than 2% (w/v) acetic acid, and optionally 0.01%-50% (w/v) polycosanol, 0.01%-5% (w/v) anti-inflammatory agent, 0.01%-10% (w/v) aloe or combinations thereof.

The aforementioned analgesic agent, anesthetic agent, combination of an astringent and an anti-infective agent and otic agents may be supplied as pure compounds, or in a form of a pharmaceutically active salt, isomer, a racemic mixture, or in any other chemical form or combination that, under physiological conditions, provided that the form still provides for therapeutically effective treatment of an otic disorder or otic disorder-related condition.

Some otic formulations include penetration enhancers to increase the therapeutic effectiveness of active agents. The term “penetration enhancer” or “permeation enhancer” refers to compounds that increase the permeability of tissue to a substance such that the rate at which the substance diffuses through the tissue and enters and bloodstream is increased. Tissue permeability may be increased by a chemical penetration enhancer via reversibly damaging or by altering the physiochemical nature of the tissue to reduce its diffusional resistance. However, although as penetration enhancers may increase the diffusion of substances through the tympanic membrane, it is not desirable that the carrier solutions and/or excipients migrate or diffuse through the tympanic membrane. Carrier solutions, if enhanced in permeating the membrane, may exacerbate an otic condition. Therefore, the use of such components in the composition is not preferred and that the above mentioned compositions be substantially free of penetration enhancers so as to minimize or prevent degradation of tympanic membrane quality and prevent or minimize fluid build up behind the membrane. The expression “substantially free of permeation enhancers” refers to an amount of permeation enhancer that is undetectable or of an amount of permeation enhancer such that migration or diffusion of the carrier solution through the tympanic membrane is negligible or not enhanced.

Examples compounds, which if used in a sufficient amount, that may function as a penetration enhancers include: low molecular weight alcohols, such as ethanol and isopropanol; polyols, such as n-alkanols, limonene, terpenes, dioxolane, propylene glycol, ethylene glycol, other glycols, and glycerol; unsaturated alkanols, such as oleyl alcohol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; and dialkylamino acetates.

A method of preventing or treating otic disorders and otic disorder-related complications in a subject that is in need of such prevention or treatment is provided, which comprises administering to the subject in need thereof a composition comprising an analgesic agent, an anesthetic agent, and a combination of an astringent and an anti-infective agent.

As used herein, the term “otic disorder” refers to any disorder or disease of the ear or post-surgical condition of the ear. Otic disorders include, for example, any condition of the ear that does not normally occur in or on the ear. Otic disorder also includes any complications that arise from having such a disorder that may develop from a prolonged untreated otic disorder. Examples of otic disorders include acoustic neuromas, acute barotitis media, acute eustachian tube obstruction, acute mastoiditis, acute otitis media, aerotitis media, aural eczematoid dermatitis, aural foreign bodies, benign paroxysmal positional vertigo, bullous myringitis, ceruminomas, chemodectomas, cholesteatoma, chronic otitis media, conductive and sensorineural hearing loss, Dandy's syndrome, diffuse external otitis, drug-induced ototoxicity, external otitis, ganglionitis, geniculate herpes, globus tympanicum tumors, herpes zoster oticus, impacted cerumen, infectious myringitis, keloids, keratosis obturans, labyrinthitis, lateral sinus thrombosis, Lermoyez's syndrome, malignant external otitis, Meniere's disease, noise-induced hearing loss, nonchromaffin paragangliomas, osteomas, otalgia, otic bleeding, otic furuncles, otic neoplasm, otic pain, otitic hydrocephalus, otitis extema, otitis media, otitis media with effusion, otomycosis, otorrhea, otosclerosis, perichondritis, perilymph fistulas, petrositis, post-otic surgery, postsurgical otalgia, presbycusis, purulent labyrinthitis, Ramsay Hunt's syndrome, sebaceous cysts, secretory otitis media, serous otitis media, squamous cell carcinoma, subdural empyema, subdural empyema and otitic hydrocephalus, subperichondrial hematoma, tinnitus, tympanic membrane infection, typanitis, vertigo, vestibular neuronitis, viral endolymphatic labyrinthitis and viral neuronitis. Preferably, the methods and compositions of the present invention comprises the prevention and treatment of the otic disorders such as otitis media, typanitis, myringitis, otitis media with effusion, otitis externa and labyrinthitis. Even more preferred the methods and compositions of the present invention comprises the prevention and treatment of the otic disorders otitis media and otitis externa.

As used herein, the term “administration” and its grammatical equivalents, when referring to use of an analgesic agent, an anesthetic agent, and a combination of an astringent and an anti-infective agent, include administration of each agent in a substantially simultaneous manner in a regimen that will provide beneficial effects of the drug combination, and includes co-administration of these agents in a sequential manner. Thus, for example, the analgesic agent and anesthetic agent, and the combination of astringent and anti-infective agent may be administered in one or more therapeutic dosage forms, such as drops or emulsions.

Sequential administration of such treatments include both relatively short and relatively long periods between the administrations of each of the agents of the present composition. Preferably, the agents are administered while at least one agent is still having an efficacious effect on the subject. Administration of each agent in a substantially simultaneous manner is generally preferred. The simultaneous presence of an analgesic agent, an anesthetic agent, and a combination of an astringent and an anti-infective agent in a subject typically has a greater efficacy than the administration of any agent alone,

When the agents are administered sequentially, it is preferred that the combination of agents are given to the subject within the therapeutic response time of at least one agent to be administered. For example, one embodiment includes the administration of an analgesic agent and anesthetic agent to the subject and the later administration of a combination of an astringent and an anti-infective agent. Preferably the combination of astringent and anti-infective agent is administered to the subject while the analgesic agent and anesthetic agent are still present in the subject at a level that is therapeutically effective.

In one embodiment, the present invention comprises a method for preventing an otic disorder in a subject, the method comprising administering to the subject an analgesic agent, an anesthetic agent, and a combination of an astringent and an anti-infective agent.

As used herein, the term “prevent” and its grammatical equivalents refer to any reduction of a subject's predisposition or risk for developing an otic disorder or an otic disorder-related complication. The term “prevent” includes either preventing a clinically evident otic disorder from occurring altogether or preventing a preclinically evident otic disorder in an individual at risk for such a disorder from occurring.

In embodiments, a method for treating an otic disorder or an otic disorder-related complication in a subject is described. The method comprises administering to the subject a composition comprising an analgesic agent, an anesthetic agent, and a combination of an astringent and an anti-infective agent.

As used herein, the term “treatment” and its grammatical equivalents refer to the alleviation or elimination of etiological or pathological symptoms and include, for example, the elimination of such symptom causation either on a temporary or permanent basis, or to alter or slow the appearance of such symptoms or symptom worsening. For example, the term “treatment” includes alleviation or elimination of causation of symptoms associated with, but not limited to, any of the otic disorders or otic disorder related-complications described herein.

As used herein, “therapeutically effective amount” refers to an amount of an active agent that is nontoxic but sufficient to provide the desired effect. The therapeutically effective amount varies according to the patient's sex, age and weight, the route of administration, the nature of the condition and any treatments which may be associated therewith, or any concurrent related or unrelated treatments or conditions of the patient. In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances. Therapeutically effective amounts can be determined without undue experimentation by any person skilled in the art or by following the exemplary guidelines set forth in this application.

Therapeutically effective refers qualitatively to the amount of an agent or agents in combination for use in an otic therapy that will achieve the goal of preventing, or improvement in the severity of, the otic disorder being treated, while avoiding adverse side effects typically associated with alternative otic therapies. An otic disorder symptom or an otic disorder-related complication symptom are considered ameliorated or improved if any benefit is achieved, irrespective of the absolute magnitude of the amelioration or improvement. For example, any reduction in pain of a subject suffering from an otic disorder such as otitis media would be considered an ameliorated symptom. Likewise, any inhibition or suppression of the normal infection and growth process for a bacterial or viral otic disorder would also be considered amelioration of an otic disorder. Furthermore, any reduction in symptom severity of an otic disorder-related complication is considered an ameliorated symptom.

As used herein, the term “subject” for purposes of treatment includes any subject, and preferably is a subject who is in need of the treatment of otic disorders, or who needs treatment of an otic disorder-related complication. For purposes of prevention, the subject is any subject, and preferably is a subject that is at risk for, or is predisposed to, developing an otic disorder or an otic disorder-related complication. The subject is typically an animal, more typically is a mammal. Preferably, the mammal is a human.

As used herein, the terms “predisposed to an otic disorder or an otic disorder-related complication” and “at risk for an otic disorder or an otic disorder-related complication,” both of which are used interchangeably herein, mean any subject at risk for developing otic disorders or any otic disorder-related complication. The subject may be a human subject who is at risk for developing otic disorders or an otic disorder-related complication. The subject may be at risk due to genetic predisposition, diet, age, exposure to a head trauma, exposure to a potentially traumatic environment, exposure to otic disorder-causing agents, and the like. The subject may also be at risk due to physiological factors such as anatomical and biochemical abnormalities in the ear. For example, children are considered at risk for developing otic disorders due to certain anatomical differences found within their ears as compared to adults.

As used herein, the terms “subject is in need of the prevention or treatment of an otic disorder or otic-disorder-related complication” refer to any subject who is suffering from or is predisposed to otic disorders or any otic disorder-related complication described herein. The terms “subject is in need of the prevention or treatment of an otic disorder or otic-disorder-related complication” also refer to any subject that requires a lower dose of active agents. In addition, the terms “subject is in need of the prevention or treatment of an otic disorder or otic-disorder-related complication” refer to any subject who requires a reduction in the side-effects of an active agent. Furthermore, the terms “subject is in need of the prevention or treatment of an otic disorder or otic-disorder-related complication” refer to any subject who requires improved tolerability to any active agent for otic disorders therapy.

In embodiments, a method of preventing or treating otic disorders and otic disorder-related complications in a subject that is in need of such prevention or treatment is provided, which comprises administering to the subject a composition comprising an analgesic agent, an anesthetic agent and a combination of an astringent and an anti-infective agent where the composition is substantially free of COX-2 inhibitors.

The method of preventing or treating otic disorders and otic disorder-related complications in a subject that is in need of such prevention or treatment may comprise administering to the subject a composition comprising an analgesic agent, an anesthetic agent, a combination of an astringent and an anti-infective agent and optionally at least one otic agent as described above. In embodiments, the method may comprise administering a composition consisting essentially of an analgesic agent, an anesthetic agent, a combination of an astringent and an anti-infective and polycosanol, and optionally an steroidal anti-inflammatory and/or aloe. The aforementioned compositions have the advantage of providing for the contacting of the tympanic membrane without substantially contributing to a build up of fluid in the middle ear. That is, the active agents are able to effectively treat the otic condition without the formulation substantially penetrating the tympanic membrane.

As used herein, the term “contacting of the tympanic membrane” refers to physical contact of the composition with at least a portion of the tympanic membrane.

As used herein, the term “contacting, without substantially penetrating the tympanic membrane” refers to reducing or eliminating the diffusion of the formulation from the external canal through the tympanic membrane to the middle ear so as to contribute to the build up of fluid behind the tympanic membrane. The active agents are able to treat the otic condition without the formulation (i.e., the carrier and/or excipients) substantially penetrating the tympanic membrane. By substantially preventing such migration across the membrane into the middle ear it is believed the etiology and pathology of many otic disorders may be reduced or eliminated.

The composition comprising an analgesic agent, an anesthetic agent, and a combination of an astringent and an anti-infective agent may be administered to a subject in need of such prevention or treatment according to standard routes of otic drug delivery that are well known to one of ordinary skill in the art. The analgesic agent, anesthetic agent, combination of an astringent and an anti-infective agent with or without the otic agents may be supplied in an acceptable topical delivery form such as solution, emlusion or the like as desirable.

The analgesic agent, anesthetic agent, combination of an astringent and an anti-infective agent with or without the otic agent can be provided in a pharmaceutically acceptable carrier or excipient to form a pharmaceutical composition. Pharmaceutically acceptable carriers and excipients include, but are not limited to, physiological saline, Ringer's solution, phosphate solution or buffer, buffered saline and other carriers known in the art. Such pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. Moreover, it is preferred that the carrier and/or excipients do not substantially penetrate the tympanic membrane. Carriers and excipients that are capable of functioning as penetration enhancers are known in the art. The compositions herein described are typically substantially absent of any penetration enhancers. The compositions herein described may optionally be supplemented with additional agents such as, for example, viscosity enhancers, preservatives and emulsifiers.

The compositions herein described are most generally preferably used as drops. Drops with a high viscosity may tend to stay in the auditory canal for longer periods and thus, may increase absorption of the active compounds by the target tissues or increase the retention time in the ear. Such viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from about 0.01% to about 2% w/v.

The topical delivery form of the composition of the method herein described is not particularly limited and may include a reservoir, a microporous rate-controlling membrane, a hypoallergenic skin contact adhesive, a priming reservoir, and a release or peel-off liner. Alternatively, the composition may be contained in a solid carrier substance, which may be in any shape, which may melt at physiological body temperature, whereby the carrier substance may be placed in the ear canal or affixed to the bottom side of a porous and flexible synthetic material which may be attached to the skin of the external ear.

Preservatives may be optionally employed to prevent microbial contamination during use of the composition. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. Typically, such preservatives are employed at a level of from about 0.001% to about 1.0% w/v.

The solubility of any of the agents of the compositions herein described may be enhanced by an appropriate co-solvent in the composition. Such co-solvents include polysorbate 20, 60, and 80, polyoxyethylene/polyoxypropylene copolymers (e.g. Pluronic F-68, F-84 and P-103), cyclodextrin, or other agents known to those skilled in the art. Typically, such co-solvents are employed at a level of from about 0.01% to about 2% w/v.

Thus, the herein described method comprises preventing and treating an otic disorder and an otic disorder-related complication in a subject in need of such prevention and treatment. The method comprises administering an amount of an analgesic agent, an anesthetic agent, a combination of an astringent and an anti-infective agent with or without an otic agent wherein the amount of the analgesic agent, anesthetic agent, combination of an astringent and an anti-infective agent, and optionally the amount of the otic agent, comprises a therapeutically effective amount.

The herein described method comprises preventing and treating an otic disorder and an otic disorder-related complication in a subject in need of such prevention and treatment may provide for minimizing or preventing degradation of tympanic membrane quality of the subject.

It will be appreciated that the amount of the analgesic agent, anesthetic agent, combination of an astringent and an anti-infective agent with or without the otic agent required for use in the treatment or prevention of otic disorders and otic disorder-related complications will vary within wide limits and may be adjusted to the individual requirements of a particular subject. The daily dosage can be administered as a single dosage or in divided dosages.

In general, for administration to adults, an appropriate daily dosage is described herein, although the limits that are identified as being preferred may be exceeded if desired. The dosage level of the amount of the analgesic agent, anesthetic agent, combination of an astringent and an anti-infective agent will necessarily depend on the particular agent that is used. It is understood that a health care provider may prepare a suitable pediatric formulation based on the appropriate daily dosage suitable for an adult, which may be determined on a case-by-case basis.

The following examples describe embodiments of the invention and are not to be construed as limiting of the scope of the invention. In the examples, all percentages are given on a weight/volume (w/v) basis unless otherwise indicated. The percent weight/volume of any component in a solution is calculated, for example, as the weight in grams of the component in a formulation multiplied by 100, divided by one milliliter volume of the formulation.

### EXAMPLE 1

An otic formulation suitable for administration to a subject may comprise 0.01-10% (w/v) benzocaine, 0.01-10% (w/v) antipyrine, 0.01-10% (w/v) aluminum acetate and 0.01-1.75% (w/v) acetic acid. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate and about 1.75% (w/v) acetic acid.

### EXAMPLE 2

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, and 0.01-50% w/v polycosanols. The polycosanols may be from plant or insect origin and may be solvent extracted or saponafied. The formulation may be prepared as an emulsion or a solution or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid and about 1% (w/v) polycosanols.

### EXAMPLE 3

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, and 0.01-5% (w/v) hydrocortisone. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid and about 2% (w/v) hydrocortisone.

### EXAMPLE 4

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, and 0.01-10% (w/v) aloe. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid and about 0.5% (w/v) aloe.

### EXAMPLE 5

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, 0.01-50% (w/v) polycosanols and 0.01-5% (w/v) hydrocortisone. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid, about 1% (w/v) polycosanols and about 2% (w/v) hydrocortisone.

### EXAMPLE 6

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, 0.01-50% (w/v) polycosanols and 0.01-10% (w/v) aloe. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid, about 1% (w/v) polycosanols and 0.5% (w/v) aloe.

### EXAMPLE 7

An otic formulation suitable for administration to a subject may comprise 0.01-10% (w/v) benzocaine, 0.01-10% (w/v) antipyrine, 0.01-10% (w/v) aluminum acetate, 0.01-1.75% (w/v) acetic acid, 0.01-50% (w/v) polycosanols, 0.01-5% (w/v) hydrocortisone and 0.01-10% (w/v) aloe. The formulation may be prepared as an emulsion or a solution, for example, in a suitable carrier suitable for use as drops to be placed in the external ear canal. Thus, an otic formulation suitable for administration to a subject may comprise about 1.4% (w/v) benzocaine, about 5.4% (w/v) antipyrine, about 1% (w/v) aluminum acetate, about 1.75% (w/v) acetic acid, about 1% (w/v) polycosanols, 2% (w/v) hydrocortisone and 0.5% (w/v) aloe.

The herein described method comprises preventing and treating an otic disorder and an otic disorder-related complication in a subject in need of such prevention and treatment by contacting, without substantially penetrating the tympanic membrane of the subject. Methods of diagnosing and monitoring the presence or change of middle ear fluid distribution, such as which occurs in an otic disorder, are generally known. By way of example, the use of a special type of otoscope, called a pneumatic otoscope, may allow the testing of tympanic membrane movement. A tympanic membrane with fluid build-up behind it does not move as well as a tympanic membrane with air behind it. Thus, the degradation of tympanic membrane quality and penetration of the membrane may be determined for compositions herein described by contacting the membrane with the compositions.

In addition, the effectiveness of a particular dosage of an analgesic agent, an anesthetic agent, and a combination of an astringent and an anti-infective agent with or without an otic agent may be determined by monitoring the effect of a given dosage on the progress or prevention of a particular otic disorder. For example, one method to detect whether, such as an ear infection, is to look in the ear a subject is suffering from an otic disorder undergoing treatment with an otoscope. By noting any reduction in redness or swelling of the tympanic membrane, which is typical of an inflammatory response, for example, due to an infection, the effectiveness of a particular dosage of an analgesic agent, an anesthetic agent, and a combination of an astringent and an anti-infective agent with or without an otic agent may be determined.

Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims.

What is claimed is:

1. A therapeutic composition for the treatment and prevention of otic disorders in humans and animals consisting essentially of:
a) benzocaine;
b) antipyrine;
c) aluminum acetate;
d) acetic acid; and
e) optionally polycosanol, an anti-inflammatory agent, aloe or combinations thereof.

2. The therapeutic composition of claim 1, wherein the anti-inflammatory agent is a corticosteroid.

3. The therapeutic composition of claim 1, wherein the corticosteroid is cortisol or hydrocortisone and admixtures thereof.

4. A therapeutic composition for the treatment and prevention of otic disorders in humans and animals comprising:
a) an analgesic agent;
b) an anesthetic agent; and
c) a combination of an astringent and an anti-infective;
wherein the composition is substantially free of COX-2 inhibitors.

5. The therapeutic composition of claim 4, wherein the anesthetic agent is benzocaine.

6. The therapeutic composition of claim 4, wherein the analgesic agent is antipyrine.

7. The therapeutic composition of claim 4, wherein the combination of an astringent and an anti-infective comprises acetic acid and at least one compound selected from the group consisting of aluminum acetate, benzalkonium chloride and benzethonium chloride.

8. The therapeutic composition of claim 7, wherein the amount of acetic acid is less than 2% (w/v) of the total composition.

9. The therapeutic composition of claim 4, further comprising polycosanol.

10. The therapeutic composition of claim 4, wherein the polycosanol comprises at least one compound selected from the group consisting of octanosol, triacontanol, behenyl alcohol, lignoceryl alcohol, ceryl alcohol, 1-heptacosanol, 1-nonacosanol, 1-dotriacontanol, and geddyl alcohol.

11. The therapeutic composition of claim 4, wherein the combination of an astringent and an anti-infective comprises at least one compound selected from the group consisting of aluminum acetate, benzalkonium chloride and benzethonium chloride.

12. The therapeutic composition of claim 4, further comprising an anti-inflammatory agent.

13. The therapeutic composition of claim 12, wherein the anti-inflammatory agent is a corticosteroid.

14. The therapeutic composition of claim 13, wherein the corticosteroid is cortisol or hydrocortisone and admixtures thereof.

15. The therapeutic composition of claim 4, further comprising aloe.

16. The therapeutic composition of claim 4, further comprising a carrier that allows administration by drops placed into the external ear canal and contact with the tympanic membrane.

17. A method for the treatment and prevention of otic disorders in humans and animals comprising:
contacting, without substantially penetrating, the tympanic membrane of the subject with a composition comprising:
a) an analgesic agent;
b) an anesthetic agent;
c) a combination of an astringent and an anti-infective; and
d) optionally with polycosanol, a steroidal anti-inflammatory or aloe,
wherein the composition is substantially free of COX-2 inhibitors.

18. A method for the treatment and prevention of otic disorders in humans and animals in need thereof comprising:
contacting, without substantially penetrating, the tympanic membrane of the subject with a composition consisting essentially of:
a) an analgesic agent;
b) an anesthetic agent;
c) a combination of an astringent and an anti-infective;
d) polycosanol;
e) a carrier, wherein the carrier allows administration by drops placed into the external ear canal; and
f) optionally an anti-inflammatory agent or aloe or combinations thereof.

19. A therapeutic otic composition comprising:
a) 0.01%-10% (w/v) benzocaine;
b) 0.01%-10% (w/v) antipyrine;
c) 0.01%-10% (w/v) aluminum acetate;
d) less than 2% (w/v) acetic acid; and
e) optionally 0.01%-50% (w/v) polycosanol, 0.01%-5% (w/v) steroidal anti-inflammatory agent, 0.01%-10% (w/v) aloe or combinations thereof.
